(19) United States
(12) Patent Application Publication
(10) Pub. No.: US 2026/0070572 A1
(43) Pub. Date: Mar. 12, 2026

(54) PROSOCIAL BEHAVIOR INTENTION PREDICTION SYSTEM AND METHOD FOR VEHICLES

(71) Applicant: Honda Motor Co., Ltd., Tokyo (JP)

(72) Inventors: Abinay Reddy NAINI, Dallas, TX (US); Zhaobo K. Zheng, Campbell, CA (US); Teruhisa Misu, San Jose, CA (US); Kumar Akash, San Jose, CA (US)

(21) Appl. No.: 18/987,498

(22) Filed: Dec. 19, 2024

Related U.S. Application Data
(60) Provisional application No. 63/693,042, filed on Sep. 10, 2024.

(51) Int. Cl. B60W 50/08; A61B 5/0205; A61B 5/024; A61B 5/0531; A61B 5/16; A61B 5/18; B60W 10/04; B60W 10/18; B60W 10/20; B60W 50/00; B60W 50/14; B60W 60/00; G08G 1/0962; G08G 1/0967

(52) U.S. Cl. CPC B60W 50/087; A61B 5/0205; A61B 5/18; B60W 10/04; B60W 10/18; B60W 10/20; B60W 50/0097; B60W 50/14; B60W 60/005; G08G 1/09626; G08G 1/096725; A61B 5/02438; A61B 5/0531; A61B 5/163; B60W 2050/146; B60W 2420/403; B60W 2540/221; B60W 2540/223; B60W 2540/30; B60W 2710/18; B60W 2710/20

(57) ABSTRACT

A method and system for implementing an action response in a vehicle based on predicted prosocial behavior of a user of the vehicle. In one embodiment, the method includes receiving real-time physiological and behavioral inputs from the user of the vehicle. The method also includes inputting the real-time physiological and behavioral inputs into a prosocial behavior prediction module onboard the vehicle. The method further includes using the prosocial behavior prediction module to output a prosocial behavior prediction for the user based on the real-time physiological and behavioral inputs from the user of the vehicle. In response to the output prosocial behavior prediction for the user, the method includes implementing at least one action response to a system of the vehicle.

### CROSS-REFERENCE TO RELATED APPLICATION

This application claims benefit to U.S. Provisional Patent Application Ser. No. 63/693,042, filed on Sep. 10, 2024 and titled “Self Supervised Learning Based Multimodal Prediction on Prosocial Behavior Intentions”, the disclosure of which application is incorporated by reference herein in its entirety.

### BACKGROUND

This disclosure relates generally to human-machine interactions, and in particular to a system and method for providing prosocial behavior intention predictions for users of vehicles, such as motor vehicles, personal transport devices, and other wheeled and non-wheeled vehicles, including automated vehicles.

Advances in machine learning, wearable sensing, and multimodal fusion enabled and empowered human state sensing and behavior predictions have applications in intelligent vehicles, digital health, and emotion recognition. User behavior prediction is critical for safe and smooth human-machine interaction, especially for interactions in mobility. Popular applications include takeover prediction in automated vehicles (AV), driving style preference of AV, and driver emotion prediction. This research area is becoming more prominent, with many promising results.

Prosocial behavior is a way of acting that takes into account one's actions towards others and society in general. It typically includes obeying rules and conforming to socially acceptable standards of kindness and consideration for other people. It is an emerging field to investigate prosocial behaviors, and people's motivation and tendency to conduct prosocial behaviors in mobility. Prosocial behaviors intend to benefit other people or society, such as yielding and slowing down for vulnerable road users. Users in modern society care more about well-being and harmony in on-road interactions because the rapid progress in AV and advanced driving assistance systems (ADAS) are fulfilling their needs for basic safety and comfort. Prosocial behavior intention prediction will be fundamentally helpful to encourage people to do more prosocial behaviors, and let intelligent vehicle systems assist them with it. However, there is not yet a data-driven method to predict such intention.

There is a need in the art for an improved system and method for providing prosocial behavior intention predictions for users of vehicles.

### SUMMARY

In one aspect, a method for implementing an action response in a vehicle based on predicted prosocial behavior of a user of the vehicle is provided. The method includes receiving real-time physiological and behavioral inputs from the user of the vehicle. The method also includes inputting the real-time physiological and behavioral inputs into a prosocial behavior prediction module onboard the vehicle. The method further includes using the prosocial behavior prediction module to output a prosocial behavior prediction for the user based on the real-time physiological and behavioral inputs from the user of the vehicle. In response to the output prosocial behavior prediction for the user, the method includes implementing at least one action response to a system of the vehicle.

In another aspect, a system for implementing an action response in a vehicle based on predicted prosocial behavior of a user of the vehicle is provided. The system includes a vehicle having a processor, a prosocial behavior prediction module, and a communication interface disposed on the vehicle. The communication interface is in communication with at least one of a user device or a wearable device worn by the user. The processor is configured to receive real-time physiological and behavioral inputs from the user of the vehicle and input the real-time physiological and behavioral inputs into the prosocial behavior prediction module onboard the vehicle. The processor is also configured to use the prosocial behavior prediction module to output a prosocial behavior prediction for the user based on the real-time physiological and behavioral inputs from the user of the vehicle. In response to the output prosocial behavior prediction for the user, the processor is configured to implement at least one action response to a system of the vehicle.

Other systems, methods, features and advantages of the disclosure will be, or will become, apparent to one of ordinary skill in the art upon examination of the following figures and detailed description. It is intended that all such additional systems, methods, features and advantages be included within this description and this summary, be within the scope of the disclosure, and be protected by the following claims.

### BRIEF DESCRIPTION OF THE DRAWINGS

The disclosure may be better understood with reference to the following drawings and description. The components in the figures are not necessarily to scale, emphasis instead being placed upon illustrating the principles of the disclosure. Moreover, in the figures, like reference numerals designate corresponding parts throughout the different views.

FIG. 1 is a schematic block diagram of an example embodiment of a vehicle that includes a prosocial behavior prediction module in accordance with aspects of the present disclosure;

FIG. 2 is a flowchart of an example embodiment of a method of providing prosocial behavior intention predictions for users of vehicles in accordance with aspects of the present disclosure;

FIG. 3 is a schematic view of an example embodiment of a physiological model pretraining process and a prosocial behavior prediction module training process in accordance with aspects of the present disclosure;

FIG. 4 is a representative view of an example embodiment of providing real-time feedback to a user of a vehicle based on a predicted prosocial behavior in accordance with aspects of the present disclosure; and

FIG. 5 is a representative view of an example embodiment of providing real-time driving assistance to a user of a vehicle based on a predicted prosocial behavior in accordance with aspects of the present disclosure.

### DETAILED DESCRIPTION

Methods and systems for providing prosocial behavior intention predictions for users of vehicles are described herein. The techniques of the present embodiments may be used to provide real-time feedback to a user of a vehicle based on a predicted prosocial behavior as well as providing real-time driving assistance to a user of a vehicle based on a predicted prosocial behavior. By encouraging and making users more aware of prosocial behavior when using vehicles, including manually operated vehicles, semi-autonomous vehicles, or autonomous vehicles, potential conflicts and negative effects between users of vehicles and others may be reduced.

The example embodiments are described herein with reference to a vehicle in the form of an automobile. The principles of the example embodiments described herein may be applied to any type of vehicle or mobility devices, such as electric scooters or other types or forms of personal transport devices, including powered devices, such as devices powered by electric motors or combustion engines, and non-powered devices, such as devices driven using a mechanical apparatus or manually propelled by users.

The present embodiments allow for users of vehicles to receive prosocial behavior intention predictions which may be provided to the user as real-time feedback and/or may be utilized by one or more autonomous or semi-autonomous systems in the vehicle to modify speed, steering, or routing of the vehicle. The prosocial behavior intention predictions are derived using a pre-trained physiological model and a prosocial behavior prediction module that uses physiological and behavioral data to predict a user's prosocial behavior in various situations to effectively improve prosocial behavior in mobility environments.

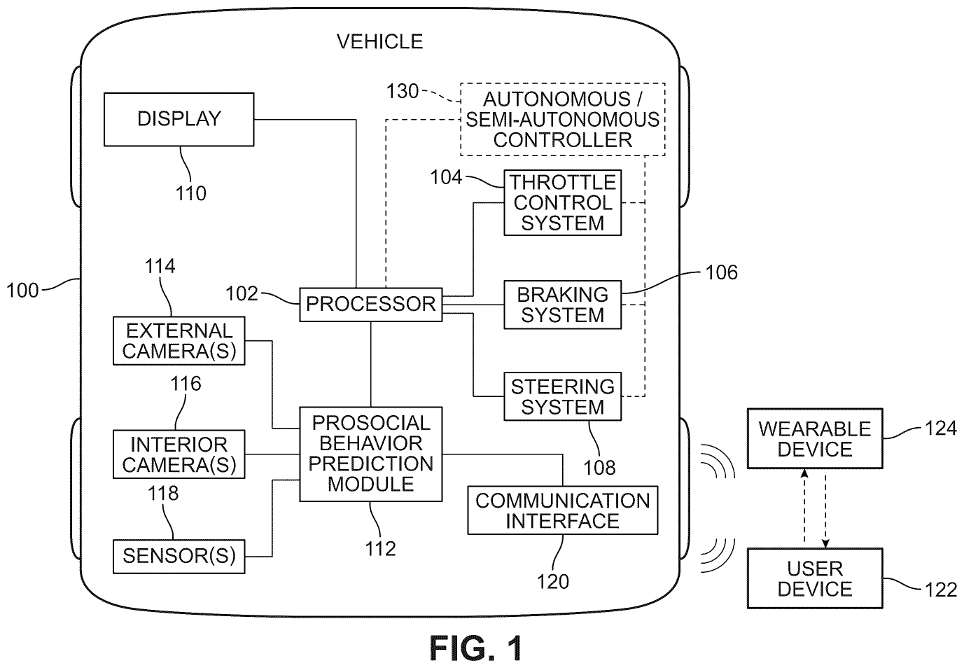

Referring now to FIG. 1, a block diagram of an example embodiment of a vehicle 100 is shown. In the exemplary embodiments described herein, vehicle 100 is shown in the form of a motor vehicle or automobile, however, it should be understood that the principles of the example embodiments may be applied to any type or form of vehicle or personal transport device, as described above.

In an example embodiment, vehicle 100 may include an onboard computing system that includes a single computing device or a network of multiple computing devices. In some embodiments, the onboard computing system may be associated with one or more electronic control units (ECUs) of vehicle 100. In one embodiment, the onboard computing system includes at least one processor 102. Processor 102 may be associated with any type of memory that may comprise a non-transitory computer readable medium. In some embodiments, instructions stored within the memory may be executed by processor 102 to implement the various functions and operations described herein.

In this embodiment, vehicle 100 includes processor 102 that receives control inputs from a user via at least a throttle control system 104, a braking system 106, and a steering system 108 to control operation of the vehicle 100. Throttle control system 104 and braking system 106 are configured to provide commands to increase and decrease, respectively, a speed or acceleration of vehicle 100. In one embodiment, throttle control system 104 may receive inputs from a user of vehicle 100 through an accelerator pedal and braking system 106 may receive inputs from a user of vehicle through a brake pedal. Steering system 108 is configured to control an orientation or direction of vehicle 100, for example, by turning one or more wheels of vehicle 100. In one embodiment, steering system 108 may receive inputs from a user of vehicle 100 through a steering wheel. In other embodiments, the specific inputs from the user to one or more of throttle control system 104, braking system 106, and steering system 108 may vary, for example, based on the type or form of vehicle or personal transport device being operated by a user.

In some embodiments, vehicle 100 may include a driver interface system that may be used to interface with the driver or other occupant of vehicle 100. To achieve this interface, the driver interface system may include input and output devices including but not limited to keyboards, touchscreens, microphones, scroll wheels, displays, speakers, and haptic systems. The driver interface system may be configured to display or otherwise present options and settings as well as other information about vehicle 100 to the user via a display 110. In this embodiment, display 110 is located within an interior cabin of vehicle 100 so that the user of vehicle 100 may view and interact with display 110. In other embodiments, display 110 may be located elsewhere and may depend on the type or form of vehicle or personal transport device.

In some embodiments, display 110 is configured to present information and real-time feedback of various parameters, including parameters associated with prosocial behavior as will be described below, to users of vehicle 100. For example, display 110 may be in the form of a screen mounted within an interior cabin or compartment of vehicle 100 that shows the user the various information captured and/or measured by cameras and/or sensors, as well as recommendations, alerts, warnings, or other real-time feedback generated by processor 102. Display 110 may also provide information to the user of vehicle 100 regarding, for example, battery life, status of lighting units, distance traveled, speed, routing and navigation information, hazard information and roadway infrastructure signals and readings.

In an example embodiment, vehicle 100 includes a prosocial behavior prediction module 112. As will be described in more detail below, prosocial behavior prediction module 112 is configured to provide prosocial behavior intention predictions to a user of vehicle 100. These prosocial behavior intention predictions provided by prosocial behavior prediction module 112 may be used to provide real-time feedback or driving assistance to a user of vehicle 100. With this arrangement, prosocial behavior in various situations may be improved in various mobility environments.

In various embodiments, prosocial behavior prediction module 112 may be implemented in software, hardware, or a combination of software and hardware. In some cases, functions of prosocial behavior prediction module 112 may be implemented using one or more processors, including processor 102, associated with vehicle 100. In other cases, one or more dedicated processors or computing devices may be provided to implement the functions of prosocial behavior prediction module 112 described herein. Additionally, in still other cases, functions of prosocial behavior prediction module 112 may be executed in part or in whole by remote computing devices, including servers, processors, and/or computing devices, in communication with vehicle 100.

In some embodiments, vehicle 100 may include components configured to detect and/or record parameters associated with vehicle 100 and the environment in which vehicle 100 is operating. In this embodiment, vehicle 100 includes one or more external camera(s) 114 configured to capture images and/or video of a scene around and exterior to vehicle 100, including in front of, to the sides of, and/or behind vehicle 100. For example, external camera 114 may capture information associated with the road, path, or route on which vehicle 100 is traveling, as well as capture information associated with objects, including people and/or other vehicles, located on or adjacent to the road, path, or route around or near vehicle 100.

In this embodiment, vehicle 100 also includes one or more additional sensors 118. In some embodiments, sensors 118 may include sensors configured to measure parameters associated with vehicle 100, a user of vehicle 100, and/or other vehicles or objects located around or near vehicle 100. For example, sensors 118 may include a GPS sensor that measures a location, speed, and heading of vehicle 100. Sensors 118 may also include types of radar or lidar that measure speed and/or distance of objects located on or adjacent to the road, path, or route around or near vehicle 100, for example, using laser or electromagnetic waves. In other embodiments, sensors 118 may also include one or more of proximity sensors, acceleration sensors, biometric sensors, occupancy sensors, steering wheel grip sensors, and vibration sensors.

In various embodiments, external camera 114, interior camera 116, and/or sensors 118 may provide processor 102 of vehicle 100 with data or parameters such as speed, distance, heading, and location associated vehicle 100 and/or objects or other vehicles located on or adjacent to the path, road, or route around or near vehicle 100. Additionally, in some embodiments, interior camera 116 and/or sensors 118 may provide processor 102 of vehicle 100 with data or parameters associated with a user of vehicle 100, such as physiological and/or behavioral data, including but not limited to information associated with gaze, pupil diameter, head movement, hand movement, arm movement, and other information associated with a body of the user.

Additionally, external road conditions (for example, adjacent vehicle proximity, dynamic objects) could be determined from a light detection and ranging system (LIDAR) and/or RADAR based sensors. In some embodiments, vehicle 100 may employ one or more of sensors 118 to generate vehicle feedback information that may be utilized by advanced driving assistance systems (ADAS) which may include, but is not limited to: vehicle lane position, relative vehicle speed, adjacent vehicle proximity, and braking response, as well as external road conditions, traffic, weather, lighting, etc. It should be understood that vehicle 100 may include other sensors known to one or ordinary skill in the art.

In some embodiments, vehicle 100 may be equipped with an autonomous or semi-autonomous controller 130. Autonomous or semi-autonomous controller 130 may include one or more types of ADAS capable of various levels of autonomous operation for systems of vehicle 100, including Level 2, Level 3, Level 4, and/or Level 5 autonomous driving as defined by Society of Automotive Engineers (SAE). For example, in this embodiment, autonomous/semi-autonomous controller 130 may control or assist with operation of one or more of throttle control system 104, braking system 106, and steering system 108 based on information received from one or more of external camera 114, interior camera 116, and/or sensors 118. In some embodiments, autonomous/semi-autonomous controller 130 may automatically implement action responses to predicted prosocial behavior from prosocial behavior prediction module 112, including automatic operation of one or more of throttle control system 104, braking system 106, and steering system 108.

In some embodiments, vehicle 100 may further include a communication interface 120. Communication interface 120 is a module that includes circuitry and software to permit vehicle 100 and components to communicate with other devices via short-range and/or long-range wireless communication technologies. For example, communication interface 120 may communicate with one or more remote computing devices over cellular or other data networks. Communication interface 120 may also communication with one or more computing devices that are located within the interior cabin or compartment of vehicle 100, such as a user device 122 and/or a wearable device 124 worn by a user of vehicle 100.

User device 122 may a mobile telephone or other mobile device owned or operated by a user of vehicle 100. Communication between user device 122 and processor 102 of vehicle 100 through communication interface 120 may be accomplished using a short-range wireless technology that allows user device 122 to communicate with vehicle 100. In an example embodiment, the short-range wireless technology may be implemented using known protocols or technologies, such as WiFi, Bluetooth®, and other types of short-range wireless or near-field communication protocols. In other embodiments, communication interface 120 may include a wired option that is directly connected to user device 122 (e.g., using a cable or dock connector).

Wearable device 124 may be a smartwatch, smart ring, or other device worn on the body of a user and having one or more sensors capable of measuring physiological information associated with the user, such as pulse rate, heart rate, breathing rate, body temperature, blood pressure, skin conductance, and other vital or biological signals from the user. In an example embodiment, wearable device 124 may communicate with processor 102 of vehicle 100 via communication interface 120. In some embodiments, wearable device 124 and user device 122 may also communicate with and between each device. In some cases, physiological data measured by wearable device 124 worn by a user may be transmitted to processor 102 of vehicle 100 through user device 122 via communication interface 120. With this arrangement, physiological data associated with the user measured by wearable device 124 may be provided to processor 102 and prosocial behavior prediction module 112.

By allowing vehicle 100 to communicate with user device 122 via communication interface 120, one or more of external camera 114, interior camera 116, sensors 118, and display 110 may be part of vehicle 100 (i.e., onboard), may be associated with user device 122 (e.g., using integrated cameras, sensors, etc.), or may be provided as a combination of onboard components and components from user device 122. In an exemplary embodiment, vehicle 100 may include a dock or other apparatus for receiving user device 122, such as a mobile device or smart phone belonging to a user of vehicle 100. With an application installed on user device 122, user device 122 may function as display 110 for vehicle 100 and can communicate with processor 102 of vehicle 100. The application on user device 122 may also monitor and/or control some of the operating systems of vehicle 100. For example, information associated with braking, speed, location, heading, turn status, etc. may be monitored via the application on user device 122.

In some embodiments, vehicle 100 may also include other components that are conventional for the type or form of vehicle or transport device being used. In the example embodiments, vehicle 100 is in the form of a motor vehicle with four wheels. In other embodiments, however, vehicle 100 may have other forms with a different number of wheels or other types of traction systems, such as tracks, treads, etc. It should be understood that the arrangement of components will vary based on the particular type and/or form of vehicle being used.

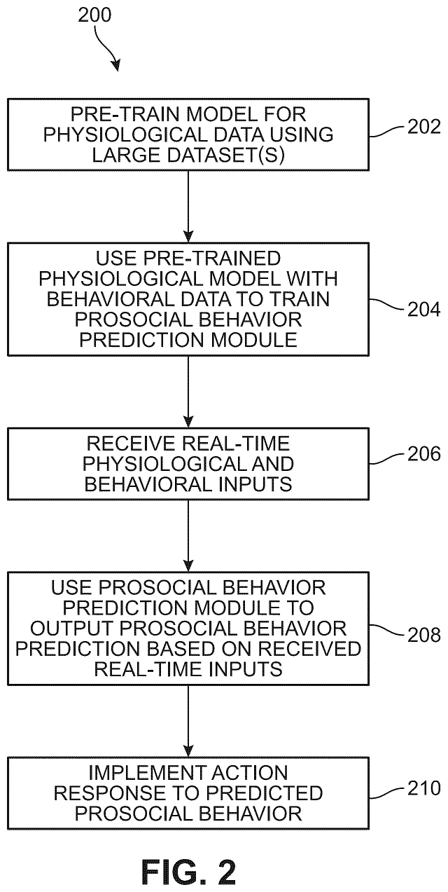

Referring now to FIG. 2, a flowchart of an example embodiment of a method 200 of providing prosocial behavior intention predictions for users of vehicles is shown. In some embodiments, one or more operations of method 200 may be implemented by processor 102 and/or prosocial behavior prediction module 112 of vehicle 100. Some operations of method 200 may be performed by other computing devices and/or processors to prepare prosocial behavior prediction module 112 for use in vehicle. In this embodiment, method 200 includes an operation 202. At operation 202, a model for physiological data is pre-trained using one or more large datasets.

For example, at operation 202, one or more large datasets for various tasks that utilize physiological data from users and sensor inputs may be used to pre-train a physiological model. In particular, while there is a shortage of long, labeled datasets specifically for prosocial behavior prediction, a wealth of datasets focus on different tasks, particularly utilizing physiological data and sensor inputs. Though not directly targeting prosocial behavior, these datasets can be invaluable in a self-supervised learning (SSL) approach, where models can pre-train on large, unlabeled, or differently labeled datasets and later fine-tune them for the specific task of prosocial behavior prediction. Such approaches have shown significant promise in related fields, enabling models to generalize well even in low-data scenarios. Accordingly, method 200 utilizes a self-supervised pretrained physiological model that leverages multi-modal datasets from various domains, including physiological data like heart rate, skin capacitance, and pupil dilation. By pre-training on diverse datasets from tasks related to emotion recognition, human state sensing, and behavioral prediction, a robust foundation is created that can then be fine-tuned with smaller, manually labeled datasets specific to prosocial behavior intention.

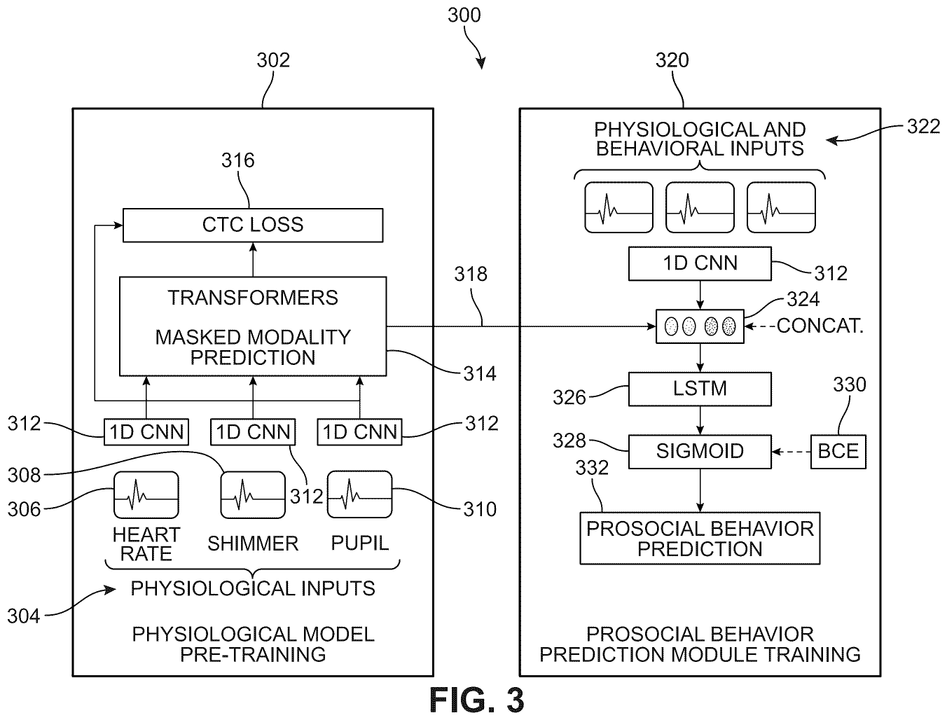

Next, method 200 includes an operation 204. At operation 204, the pretrained physiological model from operation 202 is used along with behavioral data to train a prosocial behavior prediction module (e.g., prosocial behavior prediction module 112). For example, at operation 204, a smaller dataset (e.g., smaller than the large dataset(s) used at operation 202) specifically focused on prosocial behaviors in a mobility environment was used to train prosocial behavior prediction module (e.g., prosocial behavior prediction module 112) to correlate physiological and behavioral data associated with a user to prosocial behavior intentions. Further details of operation 204 will be described below with reference to FIG. 3.

In an example embodiment, operation 202 and operation 204 may be performed or implemented one or more times to train and/or refine the physiological model and the prosocial behavior prediction module before prosocial behavior prediction module 112 is utilized within vehicle 100 for actual, real-life prosocial behavior predictions in a mobility environment. Next, operations 206, 208, and 210 of method 200 may be performed or implemented using processor 102 and/or prosocial behavior prediction module 112 of vehicle 100 after pretraining, training, and possible refinement associated with operations 202 and/or 204 has been completed.

Referring back to FIG. 2, method 200 further includes an operation 206 where real-time physiological and behavioral inputs are received. For example, at operation 206, physiological and behavioral inputs associated with a user of vehicle 100 from one or more of interior camera 116, sensors 118, user device 122, and/or wearable device 124 may be received by processor 102 and/or prosocial behavior prediction module 112 of vehicle 100. Next, at an operation 208, method 200 includes using prosocial behavior prediction module 112 to output a prosocial behavior prediction based on the real-time inputs (e.g., physiological and behavioral inputs associated with a user of vehicle 100) received at operation 206. At an operation 210, method 200 may include implementing one or more action responses to the predicted prosocial behavior determined at operation 208. For example, the action responses implemented at operation 210 may include providing real-time feedback to the user of vehicle 100 to encourage or support the user's prosocial behavior. The action responses implemented at operation 210 may also or additionally include automatic operation of one or more of throttle control system 104, braking system 106, and steering system 108 of vehicle 100 by autonomous/semi-autonomous controller 130 to implement action responses to predicted prosocial behavior.

Referring now to FIG. 3, a schematic view of an example embodiment of a training process 300 that includes pretraining physiological model 302 and prosocial behavior prediction module training 320 is shown.

FIG. 3 illustrates the architecture of pre-trained physiological model 302, which is the first step in training process 300 for generating a trained prosocial behavior prediction module (e.g., prosocial behavior prediction module 112). Physiological model 302 takes three input physiological signals or modalities 304: heart rate 306, shimmer 308 (i.e., skin capacitance), and pupil diameter 310. Each of these physiological signals or modalities 304 is passed through a dedicated one-dimensional convolutional neural network (1D CNN) 312 for each signal or modality (i.e., a separate 1D CNN 312 for each of heart rate 306, shimmer/skin capacitance 308, and pupil diameter 310) to extract low-level feature representations. With this arrangement, physiological model 302 is configured to process each physiological signal (306, 308, 310) independently.

In this embodiment, datasets with similar sensing modalities are used for self-supervised model training of physiological model 302. The datasets include: 1) takeover prediction dataset in cars and micro-mobilities, with skin conductance and gaze measurements; 2) pilot workload estimation dataset in flight simulation, with skin conductance, heart rate, and gaze measurements; and 3) trust prediction dataset with attention network. In this embodiment, these datasets are utilized because they have sensing modalities similar to those of the prediction task for the prosocial behavior prediction module.

Following the feature extraction, the processed features are fed into a four-layer regular transformer 314. This transformer 314 is designed to perform masked prediction across the available modalities 304 (e.g., heart rate 306, shimmer/skin capacitance 308, and pupil diameter 310). Specifically, during training process 300, one of the modalities is masked (i.e., one of heart rate 306, shimmer/skin capacitance 308, and pupil diameter 310), and model 302 is tasked with predicting the missing modality using the remaining two. When one modality is unavailable, it is filled with zeros and remains constant, simulating real-world scenarios with incomplete data.

To train physiological model 302, a Connectionist Temporal Classification (CTC) loss mechanism 316 that establishes positive and negative sample pairs is used. The concatenated version of the predicted and real samples from all three modalities (e.g., heart rate 306, shimmer/skin capacitance 308, and pupil diameter 310) forms a positive pair, while negative pairs are constructed by pairing each modality with data segments from different time instances. This arrangement allows physiological model 302 to learn effective representations of physiological data and prepares the pretrained physiological model 302 for downstream tasks, such as prosocial behavior intention prediction.

Referring again to FIG. 3, training process 300 for prosocial behavior prediction module training 320 to make predictions of prosocial behavior intentions utilizes the feature representations extracted from pre-trained physiological model 302 and body movement features. The first step in process 300 of prosocial behavior prediction module training 320 is a feature representation block 324, which takes output embeddings 318 from pre-trained physiological model 302 and concatenates it with body movement feature vectors 322. These body movement features 322 include head position, head rotation, and movements of the shoulders, elbows, wrists, and hands.

The body movement feature set 322 used in prosocial behavior prediction module training 320 includes both physiological and behavioral inputs, including body movement features, all Z-normalized with a moving window of 5 minutes. The physiological inputs include heart rate (1-dim), shimmer/skin capacitance (1-dim), and pupil diameter (1-dim). The body movement features include data associated with movement of the head, shoulders, elbows, wrists, and hands of the user, including: Head position (3-dim), Head rotation (4-dim), ShoulderLeft (3-dim), ShoulderRight (3-dim), ElbowLeft (3-dim), ElbowRight (3-dim), WristLeft (3-dim), WristRight (3-dim), HandLeft (3-dim), and HandRight (3-dim). Taken together, the physiological and body movement features capture essential cues for prosocial behavior prediction.

To ensure that body movement features 322 have the same dimensions as the pre-trained physiological embedding 318 from physiological model 302, body movement features 322 are passed through another 1D CNN layer 312, which normalizes and matches their dimensions. Once feature representation block 324 generates the concatenated feature vector, it is passed to a two-layer Long Short-Term Memory (LSTM) model 326. The first LSTM layer of LSTM model 326 is a sequence-to-sequence model, which processes the sequential nature of the input data (e.g., captured over 5-second intervals) and outputs an intermediate sequence.

The second LSTM layer of LSTM model 326 operates in a sequence-to-single format, reducing the sequential output from the first LSTM layer to a single prediction for each instance. In some embodiments, LSTM model 326 may be selected over a transformer layer in order to perform better in tasks with short-term dependencies, particularly in limited data settings, such as predicting prosocial behavior intentions. However, in other embodiments, LSTM model 326 may be replaced with an appropriate transformer layer. In particular, the transformer layer may be used in situations where large datasets exist for predicting prosocial behavior intentions.

Finally, the output of the second LSTM layer of LSTM model 326 is fed into a sigmoid activation layer 328, which produces a binary classification (0 or 1) representing the likelihood of prosocial behavior intention, by optimizing weighted-binary cross-entropy loss (BCE) 330. The binary classification (0 or 1) can then be used for making prosocial behavior prediction 332, with 0 representing no prosocial behavior and 1 representing a prosocial behavior. With this arrangement, training process 300 for pretraining physiological model 302 and prosocial behavior prediction module training 320 enables the completed model, embodied in prosocial behavior prediction module 112, to make accurate predictions of prosocial behavior of a user of vehicle 100.

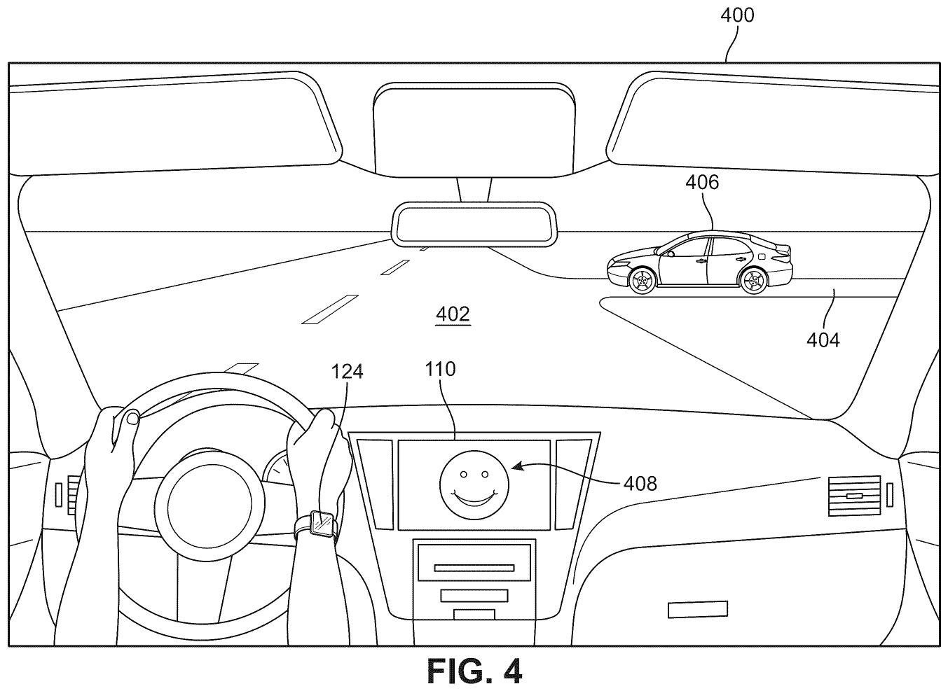

FIG. 4 is a representative view of an example embodiment of providing real-time feedback to a user of a vehicle based on a predicted prosocial behavior. In this embodiment, a first scenario 400 is illustrated where a user is traveling in vehicle 100 that includes prosocial behavior prediction module 112, described above. In an example embodiment, real-time physiological data associated with the user may be received by prosocial behavior prediction module 112 from wearable device 124 and real-time behavioral data, including one or more of the body movement features described above, may be received by prosocial behavior prediction module 112 from one or more interior cameras 116 that capture images of the user of vehicle 100.

In this embodiment, vehicle 100 is traveling forward on a road 402 and is approaching a cross street 404. At cross street 404, a second vehicle 406 is waiting to either cross road 402 or turn onto road 402. According to the techniques described herein, prosocial behavior prediction module 112 of vehicle 100 may receive the real-time physiological and behavioral inputs associated with the user of vehicle 100 to make a prosocial behavior prediction. That is, prosocial behavior prediction module 112 may use the trained model, described above in reference to FIG. 3, to predict whether or not the user of vehicle 100 is likely to take a prosocial behavior towards second vehicle 406 in scenario 400.

In the event that prosocial behavior prediction module 112 determines that the user of vehicle 100 is likely to take the prosocial behavior towards second vehicle 406, prosocial behavior prediction module 112 may implement an action response to the predicted prosocial behavior. In one embodiment, the action response implemented by prosocial behavior prediction module 112 may be in the form of providing real-time feedback to the user of vehicle 100 to encourage or support the user's prosocial behavior. For example, in scenario 400, real-time feedback 408 in the form of a smile icon or similar positive symbol or message may be presented on display 110 of vehicle 100 to the user. This real-time feedback 408 presented to the user on display 110 may serve as a reminder or encouragement to allow the user to take a prosocial behavior towards second vehicle 406, such as slowing down or changing lanes to allow second vehicle 406 to cross or turn onto road 402. With this arrangement, prosocial behavior prediction module 112 provides real-time feedback to a user of a vehicle based on a predicted prosocial behavior to encourage or support positive prosocial behavior in mobility environments.

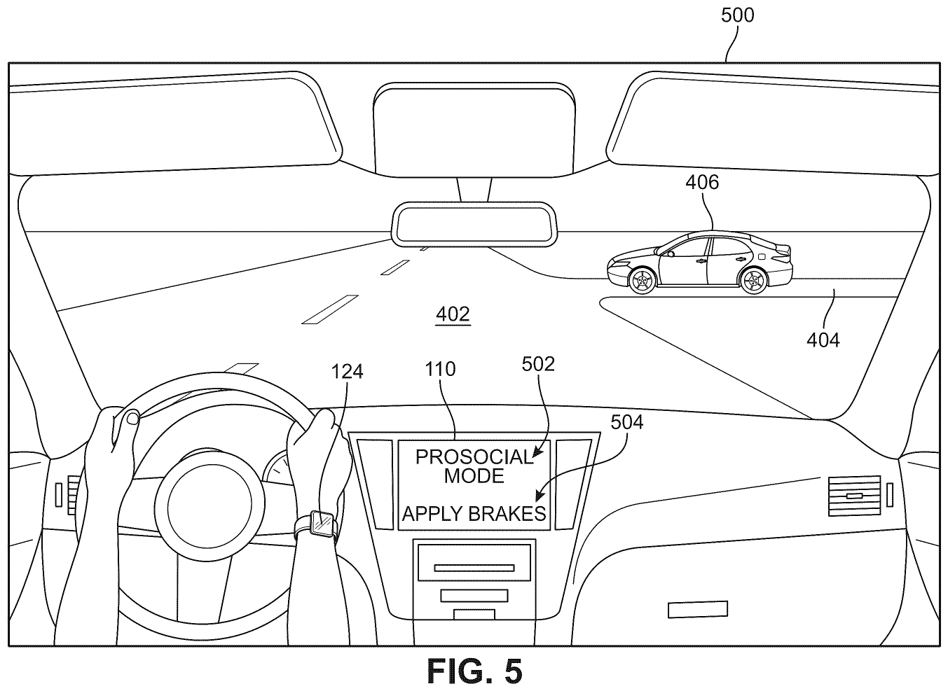

Referring now to FIG. 5, a representative view of an example embodiment of providing real-time driving assistance to a user of a vehicle based on a predicted prosocial behavior. In this embodiment, a second scenario 500 is illustrated where a user is traveling in vehicle 100 that includes prosocial behavior prediction module 112, described above, and vehicle 100 is equipped with autonomous/semi-autonomous controller 130 that may control or assist with operation of one or more of throttle control system 104, braking system 106, and steering system 108 of vehicle 100. In an example embodiment, real-time physiological data associated with the user may be received by prosocial behavior prediction module 112 from wearable device 124 and real-time behavioral data, including one or more of the body movement features described above, may be received by prosocial behavior prediction module 112 from one or more interior cameras 116 that capture images of the user of vehicle 100.

In this embodiment, vehicle 100 is traveling forward on a road 402 and is approaching cross street 404. At cross street 404, second vehicle 406 is waiting to either cross road 402 or turn onto road 402. According to the techniques described herein, prosocial behavior prediction module 112 of vehicle 100 may receive the real-time physiological and behavioral inputs associated with the user of vehicle 100 to make a prosocial behavior prediction. That is, prosocial behavior prediction module 112 may use the trained model, described above in reference to FIG. 3, to predict whether or not the user of vehicle 100 is likely to take a prosocial behavior towards second vehicle 406 in scenario 400.

In the event that prosocial behavior prediction module 112 determines that the user of vehicle 100 is likely to take the prosocial behavior towards second vehicle 406, prosocial behavior prediction module 112 may implement an action response to the predicted prosocial behavior. In this embodiment, the action response implemented by prosocial behavior prediction module 112 may be in the form of providing real-time driving assistance to the user of vehicle 100 to assist or support the user's prosocial behavior. For example, in scenario 500, real-time driving assistance in the form of a message 502 indicating engagement of a prosocial mode may be presented on display 110 of vehicle 100 to the user and autonomous/semi-autonomous controller 130 may control or assist with operation of braking system 106 by applying brakes 504 in anticipation of the user of vehicle 100 slowing down or stopping to allow second vehicle 406 to cross or turn onto road 402. In other embodiments, autonomous/semi-autonomous controller 130 may control or assist with operation of one or more of throttle control system 104, braking system 106, and steering system 108 in response to the initiation of an action response by prosocial behavior prediction module 112 when prosocial behavior is predicted. This real-time driving assistance may assist the user to take a prosocial behavior towards second vehicle 406, such as slowing down or changing lanes to allow second vehicle 406 to cross or turn onto road 402. With this arrangement, prosocial behavior prediction module 112 provides real-time driving assistance to a user of a vehicle based on a predicted prosocial behavior to encourage or support positive prosocial behavior in mobility environments.

### EXAMPLE OF THE EMBODIMENTS

A prosocial behavior intention prediction experiment was conducted using the dataset described above. Each subject participated in an hour-long session, during which they encountered opportunities for prosocial behavior in driving scenarios. For each subject, a 5-second window was considered just before a prosocial behavior encounter, labeling these windows as 1 (positive interaction) if the subject performed a prosocial action. All other window frames were labeled as 0 (negative interaction). The task for the prosocial behavior intention prediction experiment was to classify a given 5-second time frame into either 1 or 0, which determines whether the subject is likely to perform a prosocial action just after the window.

The features used for this task included physiological signals (Heart Rate, Shimmer/Skin Capacitance, and Pupil Diameter) and body movement data (such as head, shoulder, elbow, wrist, and hand positions and rotations) collected throughout the session. To assess the impact of the proposed pre-trained physiological model (e.g., physiological model 302), three different experimental setups were compared, all of which used both physiological and body movement information (referred to as All Data).

In the first case (SSL-PBIP), the proposed self-supervised learning pipeline was utilized, where the embeddings from the pre-trained physiological model 302 were concatenated with the body movement features. This combined feature set was passed to the LSTM block 326, as described above.

In the second case (LSTMPBIL), the pre-trained model 302 was bypassed and the raw physiological features (after processing through 1D CNN 312) was directly combined with the body movement features before feeding them into the same LSTM architecture.

To further explore alternative approaches, a third case (Trans-PBIL) was also considered, where the LSTM layer 326 was replaced with two transformer encoder layers to examine how transformers perform for this task.

Additionally, to explore the potential of physiological data alone, a special case (referred to as Only Physiological Data) was also considered. In this case, the same pipeline as the SSL-PBIP and LSTM-PBIL setups was followed, but zero values were provided as input for the body movement data. This allowed isolation of the impact of the physiological signals on prosocial behavior intention prediction.

For the pre-trained model 302, a 4-layer, encoder-only transformer was implemented, where each transformer layer had an embedding size of 128 dimensions for both the physiological features and body movement features (after passing through the 1D CNN 312). The model was trained on a Nvidia RTX A6000 GPU using the Adam optimizer with a learning rate of 10e-5.

To evaluate the performance of the models, two metrics were used: weighted accuracy, which accounts for class imbalance, and the F1 score, which balances precision and recall to measure the model's classification performance. The results are presented as Table 1 below.

TABLE 1

| | |
|---|---|
| All Data | Only Physiological Data |

| Metrics | WA | F1 | WA | F1 |
|---|---|---|---|---|
| LSTM-PBIP | 0.754 | 0.729 | 0.724 | 0.694 |
| Trans-PBIP | 0.743 | 0.728 | 0.718 | 0.683 |
| SSL-PBIP | 0.792 | 0.753 | 0.762 | 0.741 |

The comparison of Weighted Accuracy (WA) and F1 score for Prosocial Behavior Intention Prediction models using both All Data and Only Physiological Data. Results highlight the performance of the baseline (LSTM-PBIP and Trans-PBIP) and the proposed self-supervised learning model (SSL-PBIP).

To determine if the results are statistically significant, a one-tailed t-test was employed, considering significance at a p-value less than 0.05. This statistical analysis was crucial in confirming that the improvements across different cases were not due to random chance. From Table 1, it is evident that the proposed self-supervised learning model (SSL-PBIP) shows notable improvements over the baseline methods (LSTM-PBIP and Trans-PBIP) in both Weighted Accuracy (WA) and F1 score for the All Data case.

Specifically, the SSL-PBIP model achieves approximately 5% higher WA and over 3% improvement in F1 score compared to the LSTM-PBIP, while outperforming the Trans-PBIP model by about 6.5% in WA and around 3.5% in F1. These improvements highlight the effectiveness of leveraging a pre-trained model based on self-supervised learning (SSL) to incorporate multimodal physiological data for predicting prosocial behavior intention.

In particular, the relative performance gains for both WA and F1 metrics in the SSL-PBIP model demonstrate that pre-training with modality masking allows for better generalization when combined with body movement features. The performance difference between LSTM-PBIP and Trans-PBIP indicates that while both LSTMs and Transformers are viable architectures, the SSL-PBIP shows more than 6% better WA compared to the transformer-based approach, proving the superiority of pre-training.

Additionally, the relative improvement in the F1 score for SSL-PBIP further emphasizes that the proposed approach not only achieves higher accuracy but also maintains a well-balanced performance across both classes, avoiding overfitting to either class.

In the Only Physiological Data case, the proposed SSL-PBIP model once again achieves around 5% higher WA and about 7% better F1 score compared to LSTM-PBIP and Trans-PBIP baselines. It is particularly noteworthy that the proposed model with only physiological inputs performs better than the baseline models using all data, showing around 4.5% improvement in WA and 6% improvement in F1 score compared to the LSTM-PBIP with all data. The relatively high F1 score to that of the WA metric shows that the SSL-PBIP model is well-balanced between both classes and avoids overfitting, making it a robust solution for prosocial behavior intention prediction using physiological data alone.

The techniques described herein provide for a method and system that allows a user of a personal transport device to receive real-time feedback and/or real-time driver assistance based on predicted prosocial behavior. Integrating prosocial behavior predictions into vehicles using the techniques of the present embodiments may further help encourage users of vehicles or other transport devices to employ positive prosocial behavior towards others sharing common pathways within mobility environments.

Reference in the specification to “one embodiment” or to “an embodiment” means that a particular feature, structure, or characteristic described in connection with the embodiments is included in at least one embodiment. The appearances of the phrase “in one embodiment” or “an embodiment” in various places in the specification are not necessarily all referring to the same embodiment.

Some portions of the detailed description are presented in terms of algorithms and symbolic representations of operations on data bits within a computer memory. These algorithmic descriptions and representations are the means used by those skilled in the data processing arts to most effectively convey the substance of their work to others skilled in the art. An algorithm is here, and generally, conceived to be a self-consistent sequence of steps (instructions) leading to a desired result. The steps are those requiring physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations or transformation of physical quantities or representations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or “determining” or the like, refer to the action and processes of a computer system, or similar electronic computing device (such as a specific computing machine), that manipulates and transforms data represented as physical (electronic) quantities within the computer system memories or registers or other such information storage, transmission or display devices.

Certain aspects of the embodiments include process steps and instructions described herein in the form of an algorithm. It should be noted that the process steps and instructions of the embodiments can be embodied in software, firmware or hardware, and when embodied in software, could be downloaded to reside on and be operated from different platforms used by a variety of operating systems. The embodiments can also be in a computer program product which can be executed on a computing system.

The embodiments also relate to an apparatus for performing the operations herein. This apparatus may be specially constructed for the purposes, e.g., a specific computer, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Memory can include any of the above and/or other devices that can store information/data/programs and can be transient or non-transient medium, where a non-transient or non-transitory medium can include memory/storage that stores information for more than a minimal duration. Furthermore, the computers referred to in the specification may include a single processor or may be architectures employing multiple processor designs for increased computing capability.

The algorithms and displays presented herein are not inherently related to any particular computer or other apparatus. Various general-purpose systems may also be used with programs in accordance with the teachings herein, or it may prove convenient to construct more specialized apparatus to perform the method steps. The structure for a variety of these systems will appear from the description herein. In addition, the embodiments are not described with reference to any particular programming language. It will be appreciated that a variety of programming languages may be used to implement the teachings of the embodiments as described herein, and any references herein to specific languages are provided for disclosure of enablement and best mode.

In addition, the language used in the specification has been principally selected for readability and instructional purposes, and may not have been selected to delineate or circumscribe the inventive subject matter. Accordingly, the disclosure of the embodiments is intended to be illustrative, but not limiting, of the scope of the embodiments, which is set forth in the claims.

While various embodiments of the disclosure have been described, the description is intended to be exemplary, rather than limiting and it will be apparent to those of ordinary skill in the art that many more embodiments and implementations are possible that are within the scope of the disclosure. It is to be understood that the embodiments are not limited to the precise construction and components disclosed herein and that various modifications, changes, and variations may be made in the arrangement, operation, and details of the methods and apparatuses of the embodiments without departing from the spirit and scope of the embodiments as defined in the appended claims. Accordingly, the disclosure is not to be restricted except in light of the attached claims and their equivalents. Also, various modifications and changes may be made within the scope of the attached claims.

1. A method for implementing an action response in a vehicle based on predicted prosocial behavior of a user of the vehicle, the method comprising:
receiving real-time physiological and behavioral inputs from the user of the vehicle;
inputting the real-time physiological and behavioral inputs into a prosocial behavior prediction module onboard the vehicle;
using the prosocial behavior prediction module to output a prosocial behavior prediction for the user based on the real-time physiological and behavioral inputs from the user of the vehicle; and
in response to the output prosocial behavior prediction for the user, implementing at least one action response to a system of the vehicle.

2. The method according to claim 1, wherein the action response is providing real-time feedback to the user through a display within an interior compartment of the vehicle.

3. The method according to claim 2, wherein the real-time feedback is a message or icon shown on the display within the interior compartment of the vehicle that encourages the user of the vehicle to take a prosocial behavior towards another vehicle.

4. The method according to claim 1, wherein the action response is providing real-time driver assistance to the user of the vehicle.

5. The method according to claim 4, wherein the vehicle includes an autonomous/semi-autonomous controller configured to control or assist with operation of one or more of a throttle control system, a braking system, and a steering system of the vehicle.

6. The method according to claim 5, wherein the real-time driver assistance comprises automatic operation of one or more of the throttle control system, the braking system, or the steering system by the autonomous/semi-autonomous controller in response to the prosocial behavior prediction.

7. The method according to claim 1, wherein the real-time physiological inputs from the user of the vehicle are received from a wearable device worn by the user within the vehicle.

8. The method according to claim 7, wherein the real-time physiological inputs from the user of the vehicle include one or more of heart rate, skin capacitance, and pupil diameter detected by the wearable device.

9. The method according to claim 7, wherein the real-time behavioral inputs from the user of the vehicle are received from at least one interior camera located within an interior compartment of the vehicle.

10. The method according to claim 9, wherein the real-time behavioral inputs from the user of the vehicle include one or more body movement features associated with a head, shoulder, arm, wrist, or hands of the user detected by the at least one interior camera.

11. A system for implementing an action response in a vehicle based on predicted prosocial behavior of a user of the vehicle comprising:
a vehicle including:
a processor;
a prosocial behavior prediction module; and
a communication interface disposed on the vehicle, the communication interface being in communication with at least one of a user device or a wearable device worn by the user;
wherein the processor is configured to:
receive real-time physiological and behavioral inputs from the user of the vehicle;
input the real-time physiological and behavioral inputs into the prosocial behavior prediction module onboard the vehicle;
use the prosocial behavior prediction module to output a prosocial behavior prediction for the user based on the real-time physiological and behavioral inputs from the user of the vehicle; and
in response to the output prosocial behavior prediction for the user, implement at least one action response to a system of the vehicle.

12. The system according to claim 11, wherein at least a portion of the real-time physiological and behavioral inputs from the user of the vehicle are provided to the prosocial behavior prediction module from the user device and/or the wearable device.

13. The system according to claim 12, wherein the real-time physiological inputs from the user of the vehicle include one or more of heart rate, skin capacitance, and pupil diameter detected by the wearable device.

14. The system according to claim 11, wherein the action response is providing real-time feedback to the user through a display within an interior compartment of the vehicle.

15. The system according to claim 14, wherein the real-time feedback is a message or icon shown on the display within the interior compartment of the vehicle that encourages the user of the vehicle to take a prosocial behavior towards another vehicle.

16. The system according to claim 11, wherein the action response is providing real-time driver assistance to the user of the vehicle.

17. The system according to claim 16, wherein the vehicle includes an autonomous/semi-autonomous controller configured to control or assist with operation of one or more of a throttle control system, a braking system, and a steering system of the vehicle.

18. The system according to claim 17, wherein the real-time driver assistance comprises automatic operation of one or more of the throttle control system, the braking system, or the steering system by the autonomous/semi-autonomous controller in response to the prosocial behavior prediction.

19. The system according to claim 11, wherein the real-time behavioral inputs from the user of the vehicle are received from at least one interior camera located within an interior compartment of the vehicle.

20. The system according to claim 19, wherein the real-time behavioral inputs from the user of the vehicle include one or more body movement features associated with a head, shoulder, arm, wrist, or hands of the user detected by the at least one interior camera.
